(19) United States
(12) Patent Application Publication
(10) Pub. No.: US 2005/0059923 A1
(43) Pub. Date: Mar. 17, 2005

(54) Fenestration with intrinsic means of selective closure incorporated to a tubular body and used in interventional cardiovascular procedures

(76) Inventor: Ricardo Gamboa, La Plata (AR)

Correspondence Address: RICARDO GAMBOA, Calle 8 No 823 entre 523 bis y 524, TOLOSA, LA PLATA, BUENOS AIRES, 1900, AR

(21) Appl. No.: 10/663,703

(22) Filed: Sep. 17, 2003

(51) Int. Cl. A61F 2/06

(52) U.S. Cl. 604/9; 623/1.35; 623/1.16; 623/1.24

(57) ABSTRACT

A first arched surface (2) having a lower end fenestration (4), protruding from a tubular body (1), intersecting it along a closed oval perimeter (5).

A second arched surface (7) placed with body (1) facing first surface (2), with an upper opening (8). Both surfaces (2,7) are related through this same perimeter (5) with identical cross sections. Body (1) and both arched surfaces (2,7) are made of a waterproof material.

Surface (7) is elastically deformed along said perimeter (5), having means capable of displacing it between two operative positions.

When both surfaces (2,7) have opposed convexities of different sign, they establish a passage (9) communicating inner tubular body (6) through said opening (8) and fenestration (4).

When surface (7) acquires a same sign convexity to first surface (2), it settles against the inside of (2) closing passage (9), fenestration 4 and opening (8), with a sealing relationship.

The present invention has as aim a fenestration with intrinsic means of selective closure incorporated to a tubular body and employed in cardiovascular interventions.

More precisely, the present invention is related to one window or fenestration performed in a tubular body, chosen between a section of the tubular conduit or stent, determining such fenestration a side derivation selectively closed and eventually, opened again reestablishing the side communication under the cardiologist supervision without the contribution of any other additional device and by means of a cardiac catheterization.

To be more precise, the present invention is a tubular conduit or covered stent for intra cardiac intervention, which has at least one fenestration, incorporating intrinsic means selectively closure of the said fenestration by means of catheterizations.

### FIELD OF APPLICATION OF THE PRESENT INVENTION

The present invention has its field of application in any cardiovascular intervention in which is necessary to put a section of the conduit or a stent for a better circulation of a blood and to practice a derivation or opening named fenestration, which then should be closed according to the interventional cardiologists criterion. In particular, one of the most possible fields of greater application of the present invention is found in cardiovascular surgery and interventional catheterization techniques in congenital heart diseases and even more particularly in pediatric cardiology.

### PREVIOUS STATE OF THE ART

In such particular situations, the cardiologist has to decide to implant a stent, which will carry the blood, or to replace the already existing ones with surgical techniques according to this art.

There are situations in which it is necessary for this stent already implanted, to have a side window or fenestration which allows the volume of a secondary derivation of blood carried for a period of time, this is carried out according to the cardiologist's criterion. Then, the cardiologist by means of an appropriate intervention makes the closure of this secondary derivation or fenestration.

As an example we can mention that there are different congenital anomalies which have only one ventricle in the heart. This situation causes severe disorders in the child and in most cases, if there aren't any surgical interventions it causes death, this compels to establish a therapeutic strategy which allows the development of a special hemodynamic model.

For this reason, several surgical techniques have been develop in the last decades. These techniques tend to prepare in various stages the circulatory system making possible a correct oxygenation of the blood. That is to say, the surgical techniques allow the circulatory system to connect the venous return through the superior and inferior vena cava with the pulmonary arterial circulation.

In all cases, this implies to perform a by pass to the right ventricle.

The congenital heart with univentricular physiology which requires this type of interventions, are the following:
Single ventricle
Tricuspid atresia
Hypoplastic left heart syndrome (HLHS)
Pulmonary atresia and septum intact
Transposition of the great arteries and noncommitted ventricular septal defect and/or hypoplastic right ventricle
Criss cross heart
Congenital right ventricular hypoplasia
Double oulet right ventricle with poor anatomy
Ebstein's malformation (in some of its variants)

For example when the patient is born with single ventricle and severe pulmonary stenosis or atresia, a Blalock-Taussig shunt should be made, with a Gore-Tex tube of 4 mm from the subclavian artery to the homolateral pulmonary branch, even on the left side. In patient without pulmonary stenosis, a banding of the pulmonary artery should be performed and limited the pulmonary flow so as to avoid pulmonary hypertension which will stop the next stages.

In any of the two conditions described, the Bidirectional Glenn procedure (BDG) is performed between the ages of 6-8 months (end to side anastomosis between superior vena cava and right pulmonary artery). This technique is carried out under cardiopulmonary bypass (CB).

The last step in the patients recovery consists in undergoing the total cavo pulmonary connection (TCPC) at the age of 3 or 4, carrying the blood of the inferior vena cava to the pulmonary artery, this stage is also performed under extracorporeal cardiopulmonary bypass.

The surgical techniques have been modified in the last decades, particularly in this step. Since the original Fontan-Kreutzer procedure which joined the right atrium to the right pulmonary branch (atriopulmonary connection, APC) up to the last technique: extra cardiac conduit.

The anastomosis with the implantation of an extra cardiac conduit consists of joining the inferior vena cava to the right pulmonary branch with an interposition of a conduit, which in fact consists of an expanded polytetrafluoroethylene (PTFE) to which a fenestration is performed to the right atrium which serves as “unloading” (right-to-left shunt) to ensure the cardiac output in the postoperative period. None of these solutions have proved to have optimal results, requiring new interventions in a large number of patients, most of them performed under extracorporeal circulation.

This has been a short discussion of one of the most common heart diseases and their conventional treatment, which allows to establish with more accuracy one of the possible fields of application of the present invention; without meaning that what has been expressed up to now could be considered an application field limitation of the present invention.

Lately non surgical techniques have been performed using several stents.

As it is known, an intravascular stent, which consists in a tubular mesh of different materials such as stainless, nickel-titanium, platinum-iridium and covered with a polymeric such as expanded polytetrafluoroethylene (PTFE).

With such devices the inferior vena cava is connected to the superior vena cava, before a surgical preconditioning in the Bidirectional Glenn procedure (BDG).

The known stents used in this intervention have one or more fenestrations or openings like windows, which allow a right-to-left shunt at the atrial level, so that to maintain the cardiac output at the expense of oxygenation when conditions which limit pulmonary flow are present. After some time and according to the cardiologist criterion, these fenestrations or windows have to be closed.

The fenestration closure was usually performed using the devices available to close atrial septal defects (ASD) or patent ductus arteriosus (PDA), and needed a new intervention.

The concepts expressed up to now show as a summary of one of the possible intervention in which it is desired to have a tubular conduit or stent which could have an opening of secondary derivation or fenestration and that could be able to be opened or closed according to the cardiologist criterion through catheterization techniques without having to use any other additional device for closing the fenestration.

Up to now, it is not known in the art a tubular conduit or a stent with such characteristics, abilities and advantages to this type of interventions and others in which those conduits and/or stents could be applied.

### OBJECTS OF THE PRESENT INVENTION

The main purposes of the present invention is a piece of the tubular body or conduit opened in both ends and interpolated in a circuit system of corporal fluids (blood) and having at least a side opening or fenestration which gives way to the blood flow in derivation respect to the main volume which flows through the said tubular body and incorporating this body in cooperation with the fenestration to intrinsic means for its selective closure without requiring the use any other device. It is the purpose of the present invention that such tubular body which consists of a covered stent with fenestration in its side wall, having the intrinsic means of selective closure of such opening or closing by easy maneuver in catheterization laboratory, avoiding a new surgery.

It is another purpose of the invention, if according to the cardiologist criterion, the already closed fenestration should be re-opened, such opening and its later closing could be performed on the intrinsic means displayed in the interior of the stretch of tubular body through catheterization.

### BRIEF REVIEW OF THE INVENTION

Fenestration with intrinsic mechanism of selective closure incorporated to a tubular body and employed in interventional procedures, on which side has at least one opening which defines a fenestration for the derivation of the blood flow from the interior of the tubular body to the outside of it, characterized because the fenestration is inscribed on a piece of the wall of the tubular body which defines a first surface arched of protruding convexity projecting towards outside from the said piece of wall, with its mayor axis situated according to a generatrix of the side wall and situated between both ends of the tubular body; this first arched surface determines in its intersection with the side surface of the tubular body from which it projects a perimeter of an oval shape and the fenestration is inscribed in the inferior end. This first arched surface and fenestration are covered internally the tubular body by a second arched surface situated between the interior of the tubular body and the first arched surface, being inner part both the first and the second arched surface only related between them through the same oval perimeters in the tubular body, presenting both surfaces mutually confronted in a same development and identical transversal sections. This second arched surface has an opening inscribed inside the same oval perimeter, which communicates the inner part of the tubular body with the space defined between both arched surfaces but situated in its opposite end of the fenestration of the first surface. This first surface projects outside of the tubular body and stays immovable and that keeps its shape. The oval joint defined between both surfaces defines a zone of hinge for the second arched surface with respect to the first, being this second arched surface elastically deformed surrounding the referred perimeter, retaining two operative steady positions with elastic recovering in its previous shape. In the first of the said operative positions the second arched surface projects its convexity towards the interior of the tubular body, obtaining a convexity of opposite sign to the first arched surface and establishing a passage for the flowing of the corporeal fluid between both surfaces and communicating the interior of the tubular body through the said opening in the said passage, which communicates with the referred to fenestration, being all the sections of both arched surfaces in this way confronted the same and of opposite sign; in the second position the said second arched surface obtains a convexity of the same sign of that of the first surface and settles against the interior surface of the first surface determining the closing of the passage between both surfaces in relation to a seal and closing the fenestration and the said window in their respective surfaces; having in the interior of the second surface a means capable of displacing the second arched surface between its two mentioned operative positions, being the referred to tubular body and both arched surfaces made of a waterproof material to avoid the flowing of the corporeal fluid.

### EXAMPLES OF THE PREFERRED EMBODIMENT

To show the examples of preferred realization of the present invention, the following drawings are enclosed, with the support of the description of them given later on, to understand these examples as one of the various possible constructions of the invention, so it cannot be assigned any restrictive value, including the scope of its protection the possible equivalent means to the ones shown illustrated being the scope of the present invention determined by the first claim enclosed in the corresponding chapter of claims in this way, in these figures, the same references identify equal means and/or equivalent ones.

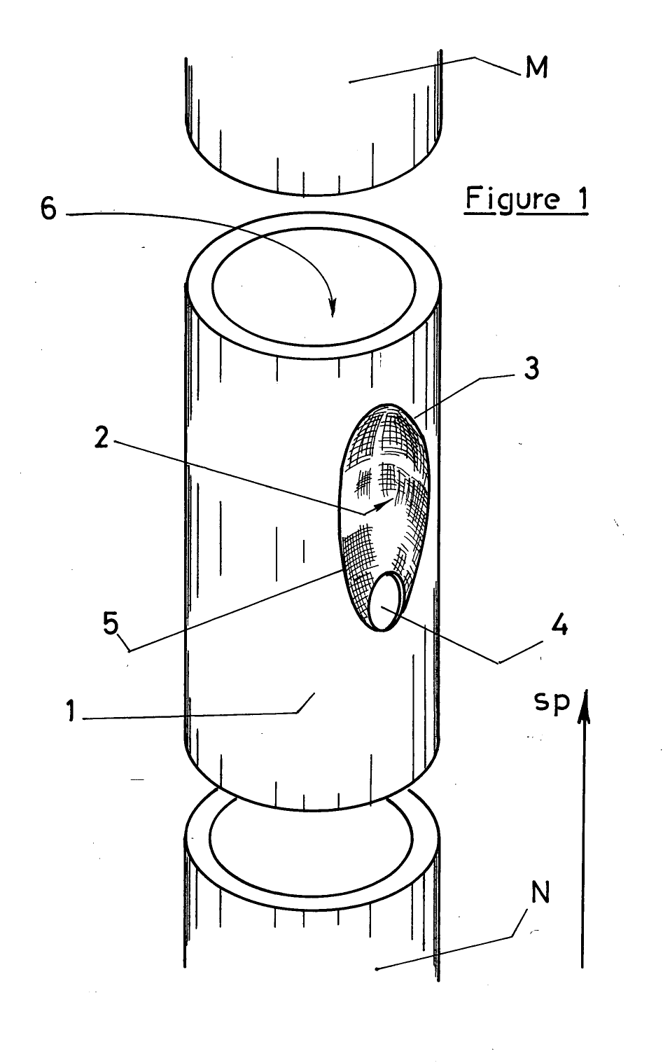

FIG. 1 shows in a superior perspective a realization which consists of a section of the tubular conduit with the closure means of the invention seen from outside and in a first plane

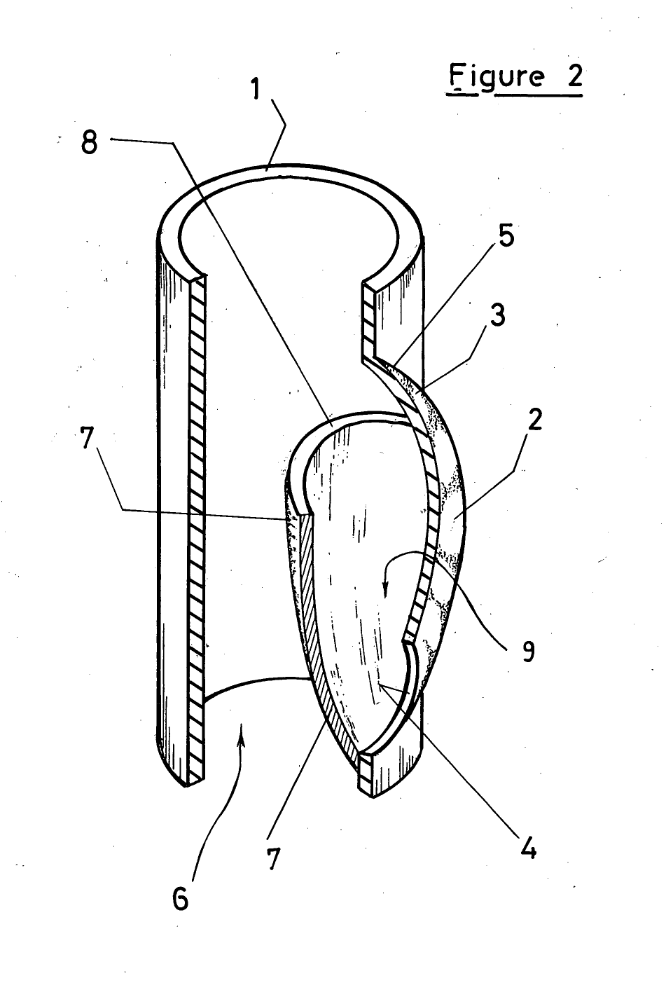

FIG. 2 shows a longitudinal cut of the realization of FIG. 1 in which the closing device can be seen in its inner part, seen from outside.

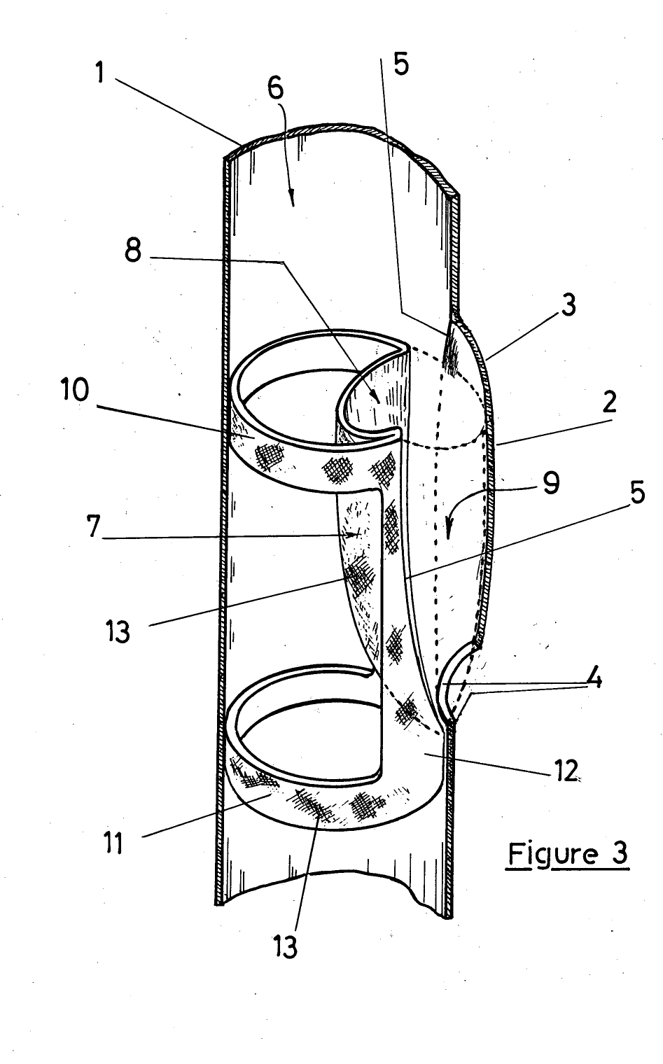

FIG. 3 shoes in detail pieces of one of the possible constructions for the realization of FIGS. 1 and 2.

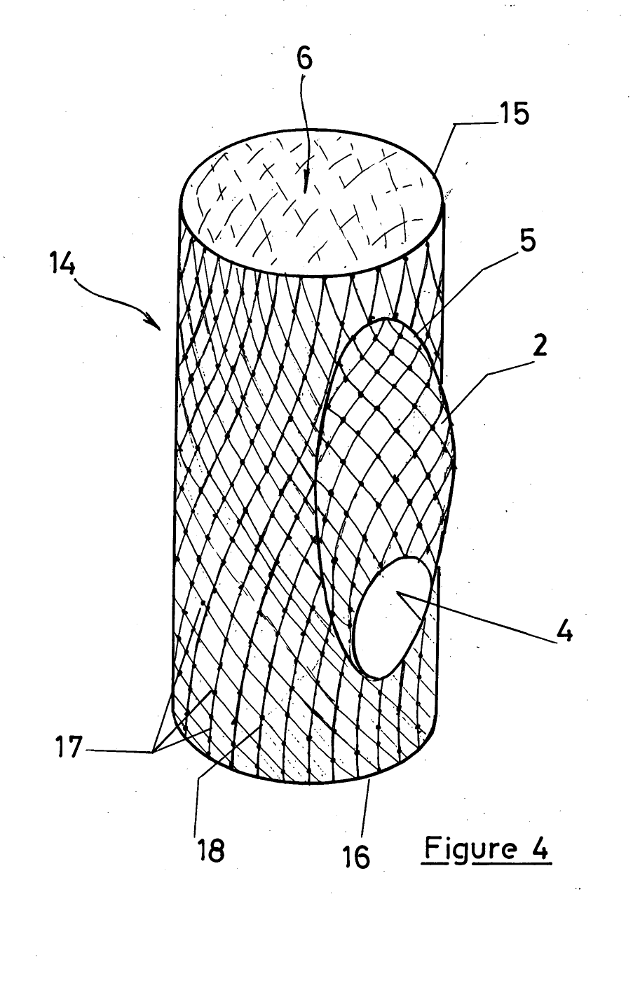

FIG. 4 illustrates another realization of the invention, which consists in a stent with closure means of the invention seen from outside and in a first plane.

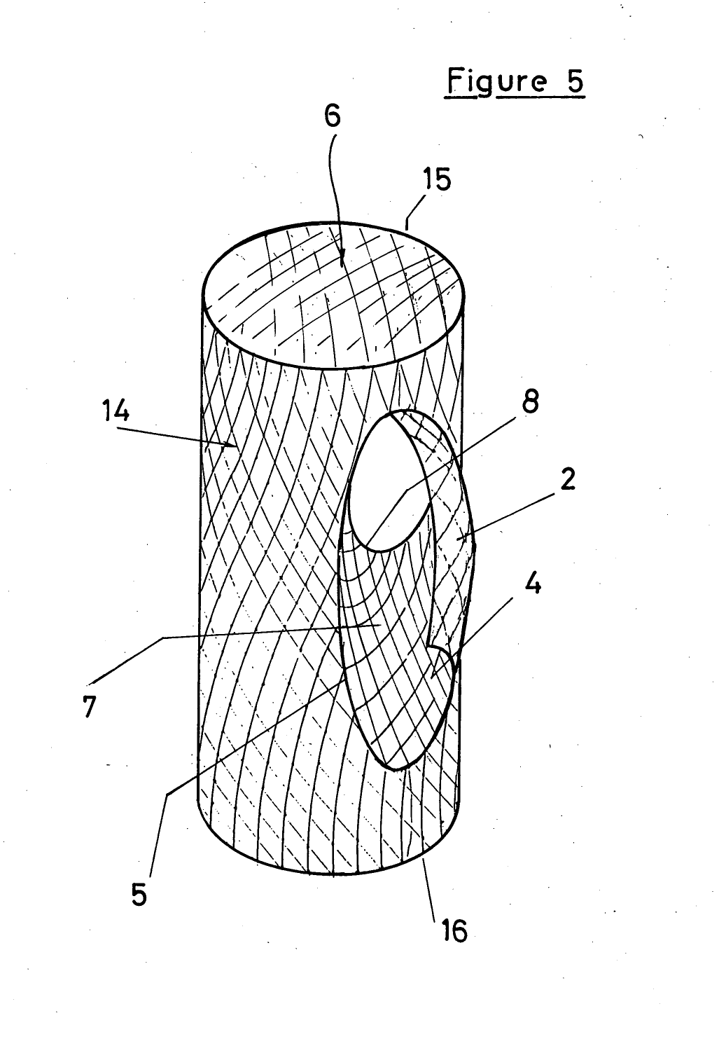

FIG. 5 shows a longitudinal cut of the realization of FIG. 4 in which the closing device can be seen in its inner part, seen from outside.

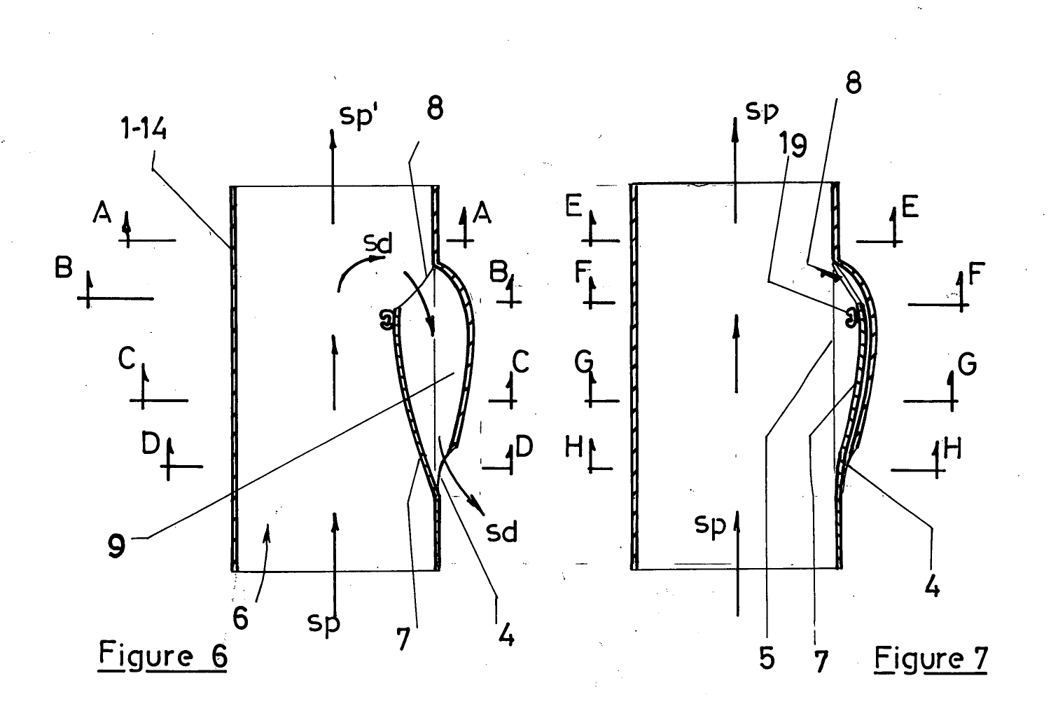

FIG. 6 allows to visualize the device of the invention, seen schematically in a diametrical longitudinal cut, with the closure means in its open position, that is to say, with the flow of the fluid of blood open.

FIG. 7 shows the same representation of picture 6, illustrating the closure means in its closing position, that is to say, closing the flow of the fluid of blood.

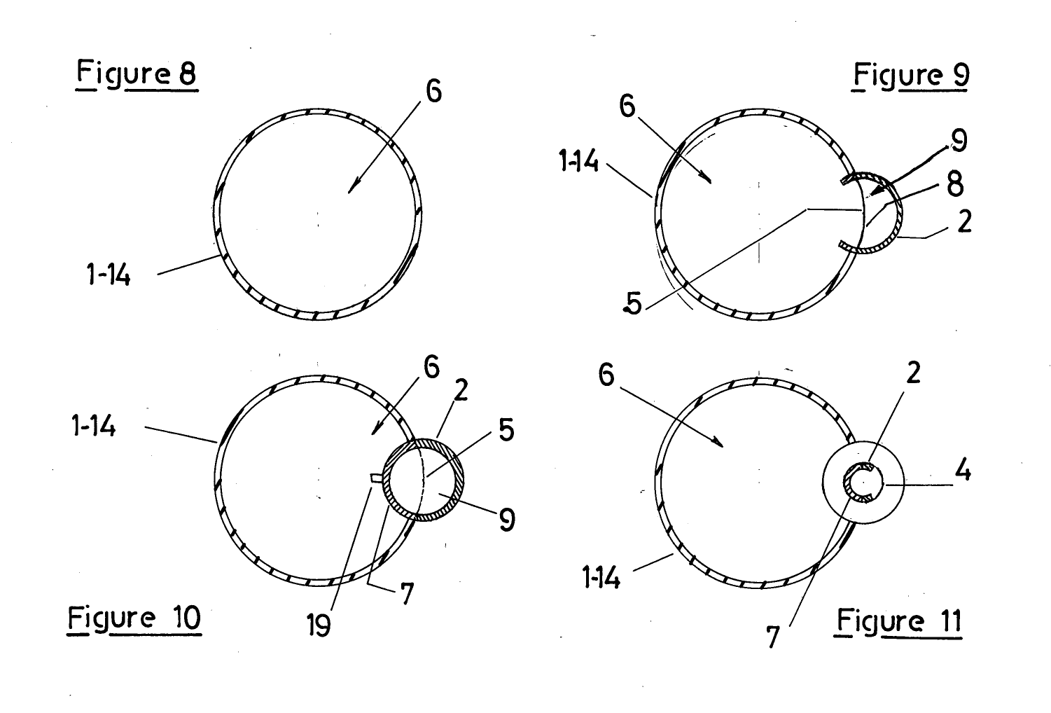

FIGS. 8, 9, 10 and 11 show respectively the diametrical cuts AA, BB, CC and DD of FIG. 6.

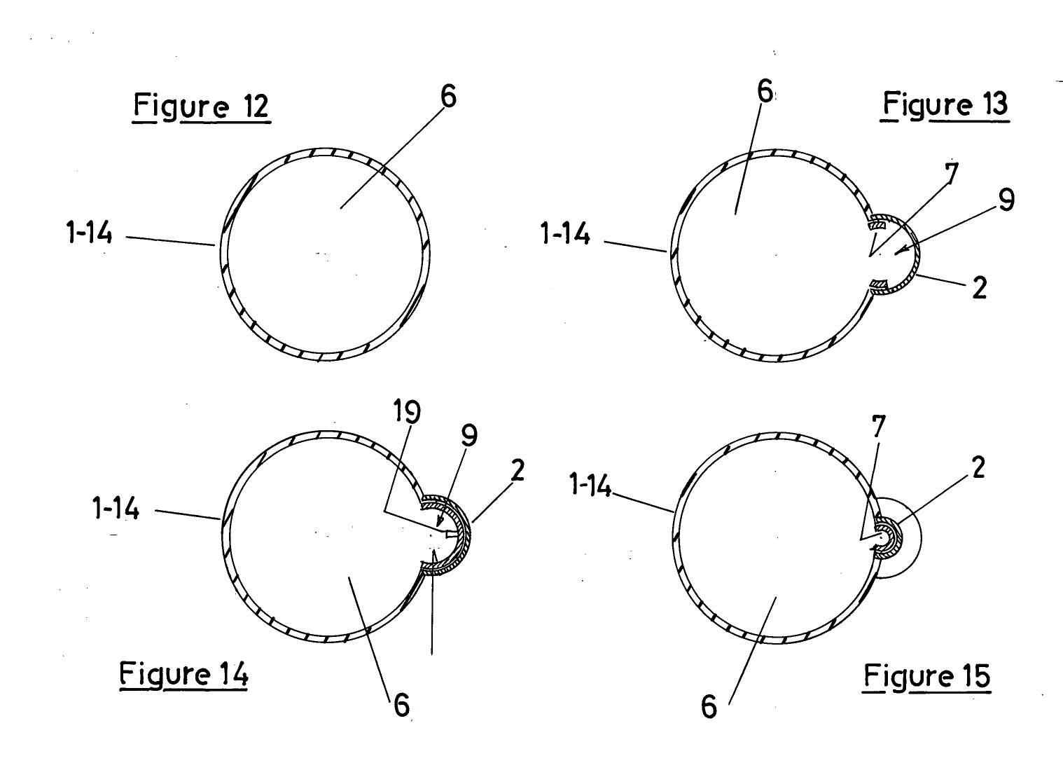

FIGS. 12, 13, 14 and 15 show respectively the diametrical cuts EE, FF, GG and HH of FIG. 7.

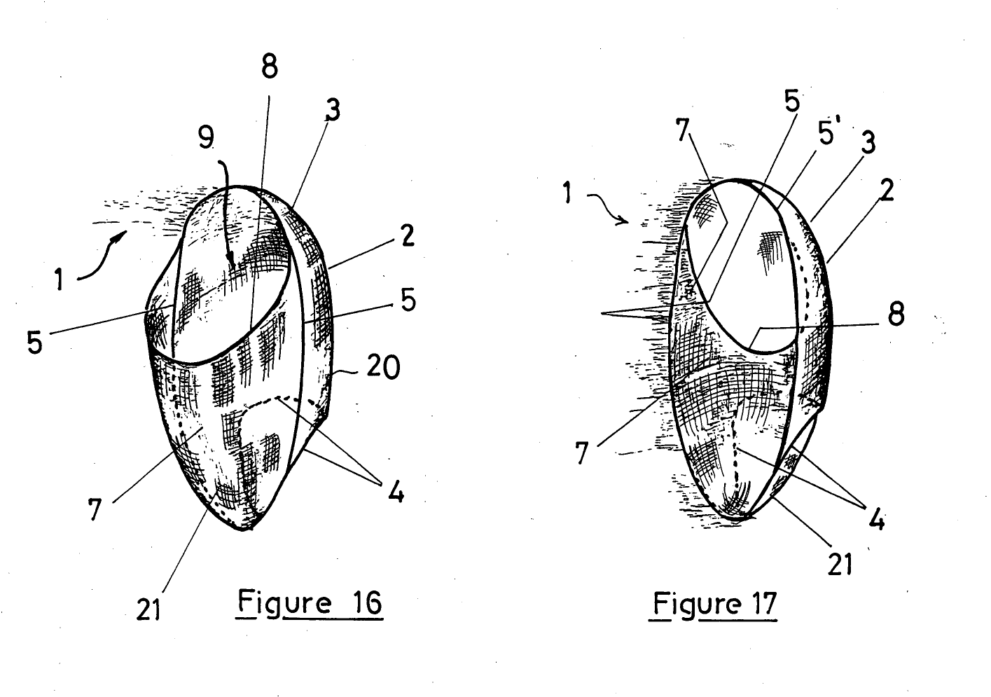

FIG. 16 illustrates the same device of FIG. 1 or 4, but seen from the interior of the stent of the invention, from a simplified perspective and partially cut, in which the device is shown with the open passage.

FIG. 17 shows the same object of FIG. 16 but showing the closed passage, seen from inside the tubular body.

In the figures the tubular body is marked with a reference (1) it consists of a conduit or section of generic tube, with a longitudinal axis, and transversal section substantially uniform.

This tubular body may consist of a short section, such as the one illustrated in the figure, capable of being incorporated in the end to end of another conduit M and N or a section (1) directly joined to arteries and/or own vessels of the patient, or else, a tubular conduit of major length. In any of these tubular pieces the present invention pretends to achieve in one side of the tubular conduit (1) a first arched surface (2) projecting or protruding towards the outside of the said conduit. The material of this conduit, as a general rule, consists of a polymeric material very particular, such as the expanded polytetrafluoroethylene (PTFE) which supplies a “supple” tube, flexible, capable of being cut and sutured without tearing, totally waterproof, and which is tolerated by the patient without being rejected.

Obviously, any other polymeric that has these same characteristics is useful to the aims of this invention. This first protruding arched surface (2) is an arched expansion in the shape of a dome, substantially enlarged and situated along one of the generatrixes of the said tube. The superior part of (2) is closed defining a hermetic dome (3), while the inferior piece of (2) shows the fenestration (4), which is an orifice through which flows the derived corporal fluid (or blood), being this hole joined to the organ or conduit of the patient according to the surgeon's criterion. The present invention expects that this orifice (4) can have a short tubular protruding length in derivation (not illustrated), or else that the surgeon directly sutures the derivation over the perimeter (4).

The first protruding arched project outside the side of the conduit (1) surrounding the oval and closed perimeter (5), being the fenestration (4) included within this perimeter (5). The material of this first protruding (2) can be the same as the material of the tube (1) or could be a modified polymeric.

The important condition is that the first arched surface defines a shell in the shape of dome with the necessary mechanic rigidity such as not to change shape under the demands of force. That is to say, this first arched surface (2) is resistant and can consist in a monolithic deformation of the walls of (1).

In the second figure it is observed, in perspective the interior of the construction of this example of the invention according to FIG. 1, showing that the first arched surface (2) is a deformation of (1), monolithic to this and that in the interior (6) of the tubular conduit (1) a second arched surface is situated (7), directly confronted to (2), placed over the same generatrix of (2) having each of the transversal sections of (7) an identical development with respect to the corresponding transversal sections of (2).

The direction of the corporeal fluid through the conduit (1) is indicated with the arrow “sp” in FIG. 1. In FIGS. 1 and 2 it is observed that the fenestration (4) is placed inscribed inside the oval perimeter (5) of the first arched surface (2) in its inferior end, that is to say, downstream respect of (2). The said second arched surface (7) is totally closed over the said perimeter (5) and has an only opening (8) inscribed in the same oval perimeter (5) which communicates the interior (6) of the tubular body with the space (9) defined between both arched surfaces (2,7), but situated in its end of the fenestration (4), is disposed upstream respect of the flow “sp” of the corporal fluid inside the tubular body (1).

Thus, according to the present invention, the exterior arched surface (2) shows a communication (4) which is opposed to a piece of continuous wall and without any openings of the second surface (7) and the opening (8) of (7) is similarly confronted to a continuous wall and without any openings of (2).

The second arched surface is made of a resistant material non deformable to keep up your arched shape in the two extremes positions, such to see in FIGS. 6 and 7, deformed surround the perimeter (5). Naturally the wall of conduit (1) and both surfaces (2,7) are impermeable.

As it has already been said, in the construction of FIGS. 1 and 2, the conduit (1) and the first surface (2) can be made of a polymeric material, of a same component or different components, of equal or different morphologic and clear nature, as far as it respects the rules of the invention above explained. The same compromise can be adopted for the second arched surface (7). This surface can be the same polymeric compound or of a different compound as the one of the surface (2). If (1) and (2) have the same polymeric composition, it is possible then to achieve that (2) consist of a monolithic expansion of (1) while if they are of a different polymeric composition, or include other components, as will be seen later, then (2) can be perimetrical joined or welded in (5) to (1) and similarly (7) can be welded internally to (5). The profile of the mouth of the entry of (8) is preferred to make a curve up as it is shown in picture 3, because of the turbulence of the fluid when it goes into the passage (9) but constructions can be seen where the edge of (8) is straight as shown in FIG. 2.

The present invention expects that this piece (2) can be stiffened modifying the crystalline structure of the polymeric and if this were not possible, another polymeric compound may be used more rigid and elastic. In consequence, either materials of different nature (such as polymeric of greater elasticity) are used for the present invention to make both arched surfaces or in at least part of the components of the invention such as the surfaces (2,7) a layer of polymeric reinforced with threads or mesh of an elastic material preferably incorporated inside the mass of the polymeric.

Another inconvenient to solve if the polymeric of the conduit or tube is too soft, is the collapse of the tube in the critical zone of the two arched surfaces. In fact, if the crushing is produced in the said zone the volume which occupies the second arched surface (7) in its projection towards the interior of the tube, could cause even the occlusion of it with the logical problems which derive from it.

FIG. 3 allows to visualize one of the possible solutions to the problem above mentioned. In the said figure a possible construction of the second arched surface (7) is observed, consisting in a piece in a material of major rigidity which is inserted inside the tube of PTFE, which is joined to two ring-shaped portions (10, 11) which are fixed against the corresponding portions of the inner wall of the tube (1), being these ring-shaped portions related to a portion of cylindrical surface (12) or gorge from which to adapt the second arched surface (7) defining on the intersection of the said surface (7) with the said gorge (12) to the perimeter (5) which determines the oval hinge around which to move an a pivot and changes shape elastically the second arched surface (7). The illustrated piece in FIG. 3 is conveniently a monolithic piece, joined in its interior to the inner wall of (1). In the said FIG. 3 the said piece has been represented reinforced by variety of metallic fibers or else textile (13) incorporated to its mass.

Another of the possible constructions of the present invention consists in incorporating both arched surfaces in a stent, represented in FIGS. 4 and 5.

A stent is a tubular device, made of a metallic mesh of thin fiber or metallic threads, capable of big elastic deformations. The characteristic of a stent is that it can be pleated surrounding a deflated balloon and situated through a guidewire, in an operation which is known as “crimping”. The balloon with the stent thus compressed is introduced inside the artery or vein and once in its place as the professional chooses, it is inflated and frees the stent, which gets the final shape and starts to fulfill its specific mission. The present invention protects any stent, made with any kind of mesh and which incorporates the resources already mentioned. The present invention, protects future stents not known yet and made of another materials, not yet used to these purposes, through they conform a tubular body with a mesh elastically distorted and covered with a impermeable polymeric to the passage of fluids, which could be displayed when expanded once freed of its sheath which covers it and get its final shape once put inside the patient.

In FIGS. 4 an 5 which sketches a generic stent, this device has been illustrated in its developed disposition, that is to say, already freed and put in the body of the patient. The stents on which the present invention is based comprehend a tubular body of a side wall (14), with two ends, the upper one or upstream (15) and the lower one or down-stream (16) showing the sense way of the fluid of blood that is to say, from (16) to (15). In FIG. 4 it is shown an schematic representation of the threads of the mesh (17) joined in its points of intersection (18), having to understand that this representation of the threads of the mesh is merely illustrative and does not try to put a limit to the present invention, being the mesh capable of adopting any other configuration.

This invention is related to a stent of the above described which has in its side wall (14) at least a window or fenestration (4) (see FIGS. 4 and 5). We say that it has at least one fenestration because if in most of the present applications the cardiology has the necessity of practicing only one window, nothing hinders in the future contemplate these devices with selective closure of one or more windows if applications which demand more than one fenestration.

In all the figures for practical purposes a construction with only one window or fenestration has been illustrated.

This invention is characterized in its side (14) where the fenestration or at least one window is practiced (4) for the flow passage of the corporal fluid such as blood, from the interior (6) of the tubular mesh towards its exterior, this fenestration is inscribed in a piece of the wall of the tubular mesh which defines a first arched surface (2) enlarged, situated between both ends (16, 15) of the conduit (14) of tubular mesh and its convexity projecting towards outside defining a first arched dorsum of a longitudinal axis substantially parallel to the longitudinal axis of the said tubular portion.

It should be noted that in that case this invention is applied to a stent as above defined, the first arched surface (2) may consist in a disfiguration of the same tubular mesh.

Each fenestration (4) practiced in the side of the stent is contained inside the disfiguration (2) of the tubular mesh of the stent defining this first arched surface and preferably this fenestration (4) it is arranged in the lower end and inscribed in the perimeter (5) of the said first arched surface. This first dorsum can present various shapes according to the demands of the design, but it is preferred to have the shape of an enlarged tear and with its perimeter oval or irregular elliptical.

This perimeter (5) is marked in the side (14) of the tubular mesh where the disfiguration of the first arched surface starts (2).

This first arched surface (2) together with the fenestration (4) is covered by a second wall of a metallic thread mesh, also covered with a impermeable polymer to the flow of blood and which determines a second arched dorsum (7) with an equal convexity and apposed to the first dorsum (2) projecting this second dorsum to the interior of the tubular conduit.

Both first and second arched surfaces (2,7) are related to the same perimeter (5) of the body of tubular mesh and both projections mutually confronted each other showing the same development and identical transversal sections, as shown in FIGS. 8 to 15.

The first arched surface (2) stays fixed respect to the tubular body (14) while the second surface (7) can pivot around its common perimeter (5) which defines a line of hinge, making use of its characteristics of elastic flexibility and has two operative stable positions when this portion belongs to the second arched surface (7) elastically deformed around the referred perimeter.

In the first of these operative positions, the second arched surface (7) projects its convexity towards the interior of the conduit of tubular mesh deforming the metallic mesh until it acquires a convexity of equal section and opposite sign to the convexity of the first arched surface (2).

As it has been said, the window or fenestration (4) is inscribed on the surface of the first arched surface (2) and within the perimeter (5) defined between the intersection of (2) with the mesh of the tubular body (14) next in the sense of the blood circulation according to the stent collocation, while the second arched surface (7) has an opening (8) in its opposite end to the fenestration (4) inscribed in the first arched surface (2). This opening (8) opens towards the interior of the tubular body (14).

In the first operative position, the second arched surface (7) is with its convexity projecting towards the interior of the stent and being confronted (as a mirror) to the first arched surface (2) it establishes between them the passage (9) for the flow (for example of blood) in derivation. As a consequence, in this first operative position the flow of blood (sp) which goes in the interior of the tubular conduit when facing the window (8) derives partially in (sd) through then (8) into the passage (9) and from there through the fenestration (4) of the first arched surface (2), goes outside the stent and shunting towards where the cardiology determines. The amount of blood coming out from the end upstream (4) is (sp′) being (sp′=sp−sd).

This first position is well sketched in FIG. 6 and in its cuts in FIGS. 8 to 15. It is observed in the said figures and particularly in FIG. 6 and in the cut CC o FIG. 10, how both arched surfaces (2-7) show the same curving and convexity but opposites. The exact shape of this curvature is according to the design of each stent for each pathology in particular, since the area of the entrance (8) and from the hole of exit (4) results the amount of fluid (blood) derived (sd) an also the shapes of the convexities an critical to avoid whirls, turbulences and returns of the flow.

In the said figures, it can be seen how the mouth of entrance (8) is preferred to have an edge of elliptic attack and growing up to avoid turbulences and the creation of vacuum in the edges of attack. Also, the profile of the hole of exit (4) is equally elliptical.

In the second operative position the above mentioned arched surface (7) is pushed and elastically deformed, turning the portions of the mesh in an unitary way over the perimeter (5) until it acquires a convexity of equal sign which is given by the first arched surface (2) where it recovers its original shape and rested against the interior surface of the second surface (2) settles against it and establishing a double wall with a great surface of contact determining the closing of the passage (9) between both dorsum causing with a relation of seal and closing the said fenestration (4) and the opening (8) in the second surface (7). This is possible because as the shapes of both dorsum coincident, the opening (8) is closed by the deformed mesh (7) when it recovers elastically and leaned against the inner wall of (2) and similarly, the bottom of (7) is leaned over the bottom of the inner face of (2) and closes the fenestration (4). It is also observed that the first arched surface (2) has a slight enlargement near its extreme (3) upstream confronting (8) and this is due to the necessity of assuring the sealed of both surfaces in close mutual contact to establish a good relation. This is observed in FIGS. 3-7 and in the cuts shown in FIGS. 9 and 13.

One of the main advantages of the present invention lies on in its capacity of close the passage in derivation (9) and selectively promote its liberation, reestablishing the shunt of blood through (9) displacing the second arched surface elastically (7) between both positions already mentioned.

The way bay which the closing of the fenestration (4) is operated by putting on both surfaces (2, 7) is insufflating the balloon catheter of the same diameter of the stent inside itself, by means of a simple maneuver of entering through (for example) the blood torrent by means of a catheterization, with a balloon, and dilating it inside the stent. The interior dorsum of the second arched surface when pushed by the balloon, changes its convexity and is lean against the inner surface of the first arched surface.

If the cardiologist notices that the fenestration in this way closed (4) is not good hemodynamic status it is operated with a catheter and is dragged the second surface again to its opening position, freeing the passage (9). For this, in a part of the inner surface of (7) and placed inside the tubular space (6) of the stent, there is a means of hooking such as a hook (19) which defines a means of hooking for a device capable of displacing the said first surface between its two operative positions.

Finally, another advantage equally important, is that this fenestration can, through of cardiac catheterization, being opened and/or closed as many times as the cardiologist believes are necessary.

Always within the field of the stent, it is preferred to use the type of construction based on a tissue or mesh of metallic threads by an adequate polymeric, even when metallic mesh can be used submerged in a layer of polymeric. Evidently, this gives a stent absolutely impermeable except for the passages used to that effect. The joining of hinge in the oval line (5) can be achieved either by weeding the layers of polymeric or by joining the metallic threads along the said oval perimeter. In FIG. 16 can be seen in a simplified way the second arched surface (7) seen in a first plane and with the passage (9) opened, that is to say, with the opening (8) and the fenestration (4) communicating.

FIG. 17 we can observe this same projection, but with the passage (9) closed. In these figures one of the constructions preferred for arched surfaces is noticed, it has an edge of perimetral intersection (5) which in this figure is visible in its whole. It is noticed that this perimetral edge (5) has a substantially oval and enlarged shape, in the shape of an ellipse with some irregularities since their ends are not equal. It is preferred that the upper end (5′) of (5) to be more rounded as regards the portion (3) which is slightly of major volume than the lower one (20) of the first arched surface (2) which allows a greater force of sealed hermetic closing both surfaces (7) against (2).

An interesting detail is given by the portion (21) of (7) which in its closing position, is exposed according to the fenestration (4) and is closed over its edges and with it can increase the action of the hermetic seal of the passage (9) when the module of transversal elasticity is increased and as consequence, its elastic response, and the surface increases when in contact with both surfaces between an opening (8) to the other (4).

With reference to the dimensions of the device of the present invention, the tubular body has an internal diameter of 16 to 22 mm., being the length of the major axis of the oval perimeter (5) of 15 to 20 mm. The opening (8) has a maximum transversal distance of 5 to 7 mm and the fenestration (4) a length according to the major axis of the perimeter (5) of 3 to 6 mm.

1- Fenestration with intrinsic means of selective closure, incorporated to a tubular body and used in cardiovascular interventions, in whose side exists at least an opening which defines a fenestration for a derivation of the flow stream of corporal fluid from the interior of the tubular body towards the exterior, characterized because the said fenestration is inscribed in a portion of the wall of the tubular body which defines a first arched surface of protruding convexity towards outside from the said portion of wall, defining this first surface an enlarged shell and non deformable under normal demands of force, with its major axis situated according to a generatrix of the side wall and situated between both ends of the tubular body. This first arched surface determines in its intersection with the side surface of the tubular body from which it projects, a perimeter of an oval shape and the said fenestration is inscribed in the lower end of the said first arched surface, downstream considering the sense of the flow of corporeal fluid along the tubular body. This first arched surface and the said fenestration are covered internally the tubular body by a second arched surface displayed between the interior of the tubular body and the first arched surface, being both the first and second surfaces only related between them through the same oval perimeter in the tubular body, showing both arched surfaces mutually confronted the same development and identical transversal sections. This second arched surface has an opening inscribed inside of the same oval perimeter, which communicates the interior of the tubular body with the space defined between both arched surfaces, but situated in its opposite end to the inscribed fenestration in the referred first arched surface and situated upstream respect to the sense of flow of the corporeal fluid inside the tubular body. The first arched surface projects outside the side wall of the tubular body and remains immovable and without changing shape, while the oval perimetral union defined between both arched surfaces defines a zone of hinge for the second arched surface respect to the first, being this second surface elastically deformable around the said perimeter, retaining two stable operative positions with elastic recovery of its previous shape. In the first of said operative positions, the second arched surface projects its convexity to the interior of the tubular body, being a convexity the opposite sign to the first arched surface, establishing a passage for the flow of the corporeal fluid between both arched surfaces, communicating the interior of the tubular body through the said opening with its passage, which communicates with the referred fenestration being all the transversal sections of both arched surfaces confronted equally and of opposite sign. In the second position the said second surface acquires a convexity of equal sign of that of the first arched surface and settles against the inner surface of the first surface causing the closing of the passage between both surface with a relation of a seal and closing this fenestration and this opening in its respective surfaces; having in the interior of the second surface a means capable of displacing this second arched surface between its two mentioned operative positions; being the referred tubular body and both arched surfaces made of a waterproof material to the flow of the corporeal fluid.

2- Fenestration with intrinsic means of selective closure, incorporated to a tubular body and used in cardiovascular interventions, according to what was claimed in 1, characterized because the tubular body is a tube in a polymeric material in which a piece of its side changes its shape, projecting itself towards outside the first arched surface; being the said first surface substantially rigid and without changing shape to the demands of exercise of the referred tube.

3- Fenestration with intrinsic means of selective closure, incorporated to a tubular body and used in cardiovascular interventions, according to what was claimed in 2, characterized because the referred tube is soft and a piece of it which defines the first arched surface has an elastic resistance to deformation of grader magnitude than the rest of the tube, settling the second arched surface on a structure substantially non deformable and which settles in the interior of the referred tube, in correspondence and confronting the concavity of the first arched surface.

4- Fenestration with intrinsic means of selective closure, incorporated to a tubular body and used in cardiovascular interventions, according to any of the claim 1 to 3 characterized because at least the first and second arched surface include a layer of a polymeric material, inside which a mesh of one chosen filaments is arranged among textile filaments and metallic filaments.

5- Fenestration with intrinsic means of selective closure, incorporated to a tubular body and used in cardiovascular interventions, according to what was in claimed 1, characterized because the said tubular piece includes a stent defined by a mesh of threads or metallic threads covered with a polymeric waterproof material, being the second arched surface defined by another piece of mesh of filaments or metallic threads covered by a waterproof polymeric material, determining this unit elastically deformable walls previous to the placement in the patient.

6- Fenestration with intrinsic means of selective closure, incorporated to a tubular body and used in cardiovascular interventions, according to what was claimed in 1 to 5, characterized because both first and second arched surfaces have surfaces of polymeric material mutually confronted and only related between them through the joint hermetic perimeter of the polymeric material coincident with the same oval perimeter in the tubular body, establishing the zone of hinge of the second surface respect to the referred oval perimeter.

7- Fenestration with intrinsic means of selective closure, incorporated to a tubular body and used in cardiovascular interventions, according to what was claimed in 5; characterized because both first an second surfaces are only related between them through the join of the metallic mesh along the oval perimeter of hinge, defining the continuity of the polymeric material in contact with a hermetic perimeter hinge which coincides with the same oval perimeter in the tubular body; establishing the zone of hinge of the second surface respect to the first in the said oval perimeter.

8- Fenestration with intrinsic means of selective closure, incorporated to a tubular body and used in cardiovascular interventions, according to what was claimed in 1 to 7, characterized because the means capable of displacing the second arched surface between its two mentioned operative positions and disposed in the interior of the second surface, consists in a protruding hook of this second surface and projecting in the interior of the tubular body.

9- Fenestration with intrinsic means of selective closure, incorporated to a tubular body and used in cardiovascular interventions, according to what was claimed in 1 to 8, characterized because the tubular body has an interior diameter from 16 to 22 mm, being the length of the major axis of the oval perimeter from 15 to 20 mm; the opening in the second arched surface which communicates the interior of the tubular body and the passage defined between both arches surfaces has a maximum transversal distance from 5 to 7 mm, and the fenestration in the first arched surface has a length according to the major axis of the perimeter from 3 to 6 mm; being the profile of the said opening in the second arched surface of curved shape and growing towards the superior end of the said oval perimeter.
